(19) United States
(12) Patent Application Publication
(10) Pub. No.: US 2008/0027250 A1
(43) Pub. Date: Jan. 31, 2008

(54) Catalytical production processes for making hydrohalopropanes and hydrofluorobutanes

(76) Inventors: Xuehui Sun, Swedesboro, NJ (US); Mario Joseph Nappa, Newark, DE (US); Velliyur Nott Mallikarjuna Rao, Wilmington, DE (US); Viacheslav A. Petrov, Hockessin, DE (US); Ekaterina N. Swearingen, Wilmington, DE (US); Nina Ivanovna Delyagina, Moscow (RU); Victor Filippovich Cherstkov, Moscow (RU)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/880,150

(22) Filed: Jul. 20, 2007

(30) Foreign Application Priority Data
Jul. 24, 2006 (RU) 2006126900

(51) Int. Cl. C07C 17/007

(52) U.S. Cl. 570/134; 570/181

(57) ABSTRACT

A process is disclosed for making hydrohalopropanes or hydrofluorobutanes. The process involves reacting a hydrofluoromethane with a fluoroolefin in the presence of an aluminum catalyst to produce a hydrohalopropane or a hydrofluorobutane. The hydrofluoromethane is CH₂F₂ or CH₃F. The fluoroolefin is CF₂═CF₂, ClFC═CF₂, or CF₃CF═CF₂.

### BACKGROUND OF THE INVENTION

1. Field of the Disclosure

The present disclosure relates in general to processes for the use of aluminum catalysts for the addition of hydrohalocarbons across the carbon-carbon double bond of fluoroolefins.

2. Description of Related Art

Halogenated compounds, especially fluorinated compounds, such as fluorocarbons and hydrofluorocarbons, have been widely used in the industry as refrigerants, solvents, cleaning agents, foam expansion agents, aerosol propellants, heat transfer media, dielectrics, fire extinguishing agents, sterilants and power cycle working fluids. There is a need for new manufacturing processes for the production of halogenated compounds.

Processes for the addition of dihalodifluoromethanes to fluoroolefins using aluminum chlorofluoride as a catalyst has been described in U.S. Pat. No. 5,488,189.

Processes for the addition of CHCl₂F to CF₂═CF₂ using a modified aluminum chloride catalyst has been described in U.S. Pat. No. 5,157,171. Although relatively efficient, the products of these processes are a varied mixture of chlorofluorocarbons, including the configurational isomers of hydrochlorofluorocarbons. Thus, a separation step is required to obtain a commercially viable product hydrofluorocarbon. For the production of a hydrofluoropropane or hydrofluorobutane, a halogen exchange reaction would also be required to convert the hydrochlorofluorocarbon to a hydrofluorocarbons. Typical halogen exchange reactions require SbF₅ catalysts in liquid phase reactions, or high temperatures in vapor phase reactions. Regardless, both the separation step and the halogen exchange steps currently available increase the costs and decrease the yields of the overall hydrofluoroalkane production processes. Thus, there is a need in the art for new manufacturing processes for the production of hydrofluorocarbons, particularly hydrofluoropropanes and hydrofluorobutances.

### SUMMARY OF THE INVENTION

A process has been provided to produce hydrohalopropanes or hydrofluorobutanes. The process comprises reacting a hydrofluoromethane with a fluoroolefin in the presence of an aluminum catalyst to produce a hydrohalopropane or a hydrofluorobutane. The hydrofluoromethane is CH₂F₂ or CH₃F. The fluoroolefin is CF₂═CF₂, ClFC═CF₂, or CF₃CF═CF₂.

The foregoing general description and the following detailed description are exemplary and explanatory only and are not restrictive of the invention as defined in the appended claims.

### DETAILED DESCRIPTION OF THE INVENTION

Before addressing details of embodiments described below, some terms are defined or clarified.

The term “a hydrofluoromethane” is intended to mean a partially fluorinated methane selected from the group consisting of CH₂F₂ and CH₃F.

The term “a fluoroolefin” is intended to mean a fluorinated olefin selected from the group consisting of CF₂═CF₂, ClFC═CF₂, and CF₃CF═CF₂.

The term “a hydrohalopropane” is intended to mean a propane wherein partial hydrogens are substituted by halogens. In one embodiment of this invention, a hydrohalopropane is a product of the reaction between a hydrofluoromethane and a perhaloethylene and is selected from the group consisting of CF₃CF₂CH₂F, CF₃CF₂CH₃, CF₃CFClCH₂F, CF₃CFClCH₃, CF₂ClCF₂CH₂F and CF₂ClCF₂CH₃.

The term “a hydrofluorobutane” is intended to mean a butane wherein partial hydrogens are substituted by fluorines. In one embodiment of this invention, a hydrofluorobutane is a product of the reaction between a hydrofluoromethane and CF₃CF═CF₂ and is selected from the group consisting of CH₂FCF(CF₃)₂ and CH₃CF(CF₃)₂.

The term “an aluminum catalyst” is intended to mean a catalyst with the general formula of AlCl₃₋ₘFₘ or AlBr₃₋ₙFₙ, wherein m is from about 1.0 to 3, and n is from about 2.7 to 3.

In one embodiment of this invention, an aluminum catalyst is AlF₃. AlF₃ is a known compound, and its preparation method has been disclosed, for example, by S. Rudiger, et al. in J. Sol-Gel Sci. Techn. Volume 41 (2007) 299-311, hereby incorporated by reference in its entirety.

In another embodiment of this invention, an aluminum catalyst is a modified aluminum chloride.

In another embodiment of this invention, an aluminum catalyst is a modified aluminum bromide.

The term “a modified aluminum chloride” is intended to mean an aluminum chlorofluoride containing about 3 to about 64% F by weight.

In one embodiment of this invention, the aluminum chlorofluoride contains about 16 to 61% F by weight. Such aluminum chlorofluoride can be represented by formula AlCl₃₋ₓFₓ wherein x is typically about 1.0 to about 2.8.

The term “a modified aluminum bromide” is intended to mean an aluminum bromofluoride represented by formula AlBr_{3-y}F_{y} wherein y is typically about 2.7 to about 2.95.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. Although methods and materials similar or equivalent to those described herein can be used in the practice or testing of embodiments of the present invention, suitable methods and materials are described below. All publications, patent applications, patents, and other references mentioned herein are incorporated by reference in their entirety, unless a particular passage is cited. In case of conflict, the present specification, including definitions, will control. In addition, the materials, methods, and examples are illustrative only and not intended to be limiting.

A process has been provided to produce hydrohalopropanes or hydrofluorobutanes. The process comprises reacting a hydrofluoromethane with a fluoroolefin in the presence of an aluminum catalyst to produce a hydrohalopropane or a hydrofluorobutane. The hydrofluoromethane is CH₂F₂ or CH₃F. The fluoroolefin is CF₂═CF₂, ClFC═CF₂, or CF₃CF═CF₂.

A process has also been provided to produce hydrohalopropanes or hydrofluorobutanes. The process comprises reacting a hydrofluoromethane with a fluoroolefin in the presence of a modified aluminum chloride catalyst or a modified aluminum bromide catalyst to produce a hydrohalopropane or a hydrofluorobutane. The hydrofluoromethane is CH₂F₂ or CH₃F. The fluoroolefin is CF₂═CF₂, ClFC═CF₂, or CF₃CF=CF₂.

Many aspects and embodiments have been described above and are merely exemplary and not limiting. After reading this specification, skilled artisans appreciate that other aspects and embodiments are possible without departing from the scope of the invention.

Other features and benefits of any one or more of the embodiments will be apparent from the following detailed description, and from the claims.

Modified aluminum chlorides can be prepared by reacting commercially avaible anhydrous AlCl₃ with one or more chlorofluorocarbons, hydrochlorofluorocarbons, or hydrofluorocarbons as disclosed in U.S. Pat. No. 5,157,171 to Sievert, et al., which is incorporated herein by reference. By way of explanation, the modified aluminum chloride catalysts used in the process are prepared by treating anhydrous aluminum chloride with an excess of chlorofluorocarbons, hydrochlorofluorocarbons, or hydrofluorocarbons such as CH₃F, CH₂F₂, CHF₃, CCl₂ FCCl₃, CClF₂ CCl₃, CF₃,CCl₃, CF₃ CCl₂F, CF₃ CClF₂, CHCl₂ CCl₂ F, CHClFCCl₃, CHCl₂ CClF₂, CHClFCCl₂ F, CHF₂ CCl₃, CHCl₂ CF₃, CHClFCClF₂, CHF₂ CCl₂ F, CHClFCF₃, CHF₂ CClF₂, C₂HF₅, CHClFCHCl₂, CH₂ ClCCl₂ F, CH₂ FCCl₃, CHClFCHClF, CHCl₂ CHF₂, CH₂ClCClF₂, CH₂ FCCl₂F, CHClFCHF₂, CH₂ClCF₃, CH₂FCClF₂, CHF₂CHF₂, CH₂FCF₃, CH₂ClCHClF, CH₂FCHCl₂, CH₃CCl₂ F, CH₂ClCHF₂, CH₂FCHClF, CH₃CClF₂, CH₂FCHF₂, CH₃CF₃, CH₂FCH₂ Cl, CH₃CHClF, CH₂ FCH₂F, CH₃ CHF₂, and C₂H₅F; preferably CCl₂F₂, CHCl₂ F, CHClF₂, CH₂ClF, CCl₂FCCl₂ F, CCl₂FCClF₂, CClF₂ CClF₂; and most preferably CCl₃F. It is believed that propane derivatives displaying the structural features shown above may also be used in the process of this invention. The reaction between aluminum chloride and the chlorofluorocarbons, hydrochlorofluorocarbons, or hydrofluorocarbons occurs, for the most part, spontaneously, and is exothermic. In certain instances, such as with C₂ chlorofluorocarbons, slight heating may be used advantageously. For compounds containing —CF₃ groups such as CHF₃, CCl₃CF₃, CHCl₂CF₃, CH₂ClCF₃, and CH₃CF₃ more vigorous conditions are required to effect reaction with AlCl₃, and the reaction is best carried out under the pressure developed autogenously by the reactants. After the reaction has subsided, the liquid products are removed, generally under reduced pressures to provide a modified aluminum chloride catalyst which will usually contain from about 3 to about 68% fluorine by weight. The liquid product from the reaction of chlorofluorocarbons with AlCl₃ includes products which are produced by halogen exchange reaction with the aluminum chloride as well as rearranged chlorofluorocarbons.

The solid modified aluminum chloride product of the reaction of AlCl₃ with chlorofluorocarbons may be separated from the liquid products by filtration, by distillation or vacuum transfer of the liquid products from the modified aluminum chloride, or, alternatively, the modified aluminum chloride catalyst may be used as a suspension for subsequent reactions.

Modified aluminum bromides can be prepared by reacting commercially avaible anhydrous AlBr₃ with CCl₃F as disclosed in Journal of Fluorine Chemistry 127 (2006) 663-678 by T. Krahl and E. Kemnitz, which is incorporated herein by reference.

In one embodiment of this invention, the modified aluminum chloride catalyst or the modified aluminum bromide catalyst is produced before the catalyst is contacted with reactants hydrofluoromethane or fluoroolefin.

In another embodiment of this invention, the reactant hydrofluoromethane may also be employed in the formation of modified aluminum chloride catalyst. Use of sufficient excess of reactant hydrofluoromethane enables the production of modified aluminum chloride catalyst in situ from anhydrous aluminum chloride so that the catalyst modification reaction need not be carried out as a separate step.

In another embodiment of this invention, the reactant hydrofluoromethane may also be employed in the formation of modified aluminum bromide catalyst. Use of sufficient excess of reactant hydrofluoromethane enables the production of modified aluminum bromide catalyst in situ from anhydrous aluminum bromide so that the catalyst modification reaction need not be carried out as a separate step.

In yet another embodiment of this invention, the reactants hydrofluoromethane and fluoroolefin can be simultaneously contacted with the anhydrous aluminum chloride or the anhydrous aluminum bromide.

The molar ratio of the reactant hydrofluoromethane to the reactant fluoroolefin is at least 1:1. In one embodiment of this invention, the molar ratio of the reactant hydrofluoromethane to the reactant fluoroolefin is at least 3:1. In another embodiment of this invention, the molar ratio of the reactant hydrofluoromethane to the reactant fluoroolefin is at least 5:1

Optionally, solvents may be employed in the reaction process. In one embodiment of the invention, the reactant hydrofluoromethane is also used as a solvent. In another embodiment of the invention, the solvent is an inert chemical compound and shall not react with other chemical compounds or catalysts during the reaction. Such inert solvents, if used, should boil at a temperature enabling separation from the unconverted reactants hydrofluoromethane and fluoroolefin and from the product hydrohalopropane or hydrofluorobutane. In one embodiment of the invention, a suitable inert solvent is selected from perfluorocarbons or hydrohalocarbons which will not react with other chemical compounds or catalysts during the reaction. In another embodiment of the invention, a suitable inert solvent is selected from the group consisting of CCl₄, CF₃CHCl₂, CCl₃CF₃, CF₃CF₂CH₂F, CF₃CF₂CH₃, CF₃CFClCH₂F, CF₃CFClCH₃, CF₂ClCF₂CH₂F, CF₂ClCF₂CH₃, CH₂FCF(CF₃)₂ and CH₃CF(CF₃)₂.

In one embodiment of the invention, the inert solvent is the same chemical compound as the product hydrohalopropane or hydrofluorobutane.

The temperature employed in the reaction process typically ranges from about −10° C. to 200° C. In one embodiment of the invention, the temperature employed in the reaction process ranges from about 0° C to 100° C.

Reaction time is not critical and typically ranges from about 0.25 hours to about 24 hours.

The pressure employed in the reaction is not critical. Typically, the reaction is conducted under autogenous pressure. However, the pressure should not exceed 300 psi when tetrafluoroethylene is used as an reactant.

The product hydrohalopropane or hydrofluorobutane may be recovered by filtration or fractional distillation. In one embodiment of this invention, the catalyst is decomposed by treatment with water and the product hydrohalopropane is then recovered by fractional distillation. In another embodiment of this invention, the catalyst is decomposed by treatment with water and the product hydrofluorobutane is then recovered by fractional distillation.

The reactors, distillation columns, and their associated feed lines, effluent lines, and associated units used in applying the processes of embodiments of this invention should be constructed of materials resistant to corrosion. Typical materials of construction include stainless steels, in particular of the austenitic type, the well-known high nickel alloys, such as Monel™ nickel-copper alloys, Hastelloy™ nickel-based alloys and, Inconel™ nickel-chromium alloys, and copper-clad steel.

### EXAMPLES

The concepts described herein will be further described in the following examples, which do not limit the scope of the invention described in the claims.

### Example 1

Example 1 demonstrates a method for preparing a modifiedaluminum chloride catalyst.

A 1 L four-neck flask was equipped with an addition funnel, a mechanical stirrer, a dry ice condenser and nitrogen purge. The flask was charged with AlCl₃ (75g, 0.56 mol). CCl₃F (205 g, 1.49 mol) was then slowly added to the flask under nitrogen purge through the addition funnel. The reaction was not heated. Stirring was continued for another 15 minutes after all CCl₃F had been added. The volatiles were removed in vacuum and the resulting solid modified aluminum chloride catalyst was vacuum dried at 60° C.

### Example 2

Example 2 demonstrates a reaction of CH₂F₂ with CF₂═CF₂ to produce CF₃CF₂CH₂F by using a modified aluminum chloride catalyst prepared according to the procedure described above in Example 1.

A 400 ml Hastelloy™ C shaker tube was charged with 10 g of modified aluminum chloride. The tube was cooled down to −10° C. and evacuated. The tube was then charged with 60 g CH₂F₂/CF₂═CF₂ mixture (1:1 molar ratio, 0.39 moles each). Then the reaction mixture was stirred at 60° C. for 6 hours. 34 g product mixture was collected in a cold trap and was analyzed by GC-MS. The analytical results are given in units of GC area % in Table 1 below. Small amounts of other byproducts, having GC area % less than 0.1, are not included in Table 1.

TABLE 1

| CH₂F₂ | CF₂ = CF₂ | CF₃CHF₂ | CCl₂F₂ | CF₃CF₂CH₂F | CHF₂CF₂CF₂Cl | CF₃CF₂CH₂Cl |
|---|---|---|---|---|---|---|
| 3.7 | 5.6 | 2.0 | 0.3 | 61.5 | 3.1 | 19.0 |

### Example 3

Example 3 demonstrates a reaction of CH₂F₂ with CF₂═CF₂ to produce CF₃CF₂CH₂F by using a modified aluminum chloride catalyst produced in situ.

A 400 ml Hastelloy C shaker tube was charged with AlCl₃ (16 g, 0.12 mol). The tube was cooled down to −10° C. and evacuated. The tube was then charged with 60 g CH₂F₂ /CF₂═CF₂ mixture (1:1 molar ratio, 0.39 moles each). Then the reaction mixture was stirred at 60° C. for 6 hours. 60 g product mixture was collected in a cold trap and was analyzed by GC-MS. The analytical results are given in units of GC area % in Table 2 below. Small amounts of other products, having GC area % less than 0.1, are not included in Table 2.

TABLE 2

| CF₃CHF₂ | CHF2CHCl2 | CF₃CF₂CH₂F | CF₃CF₂CH₂Cl |
|---|---|---|---|
| 0.6 | 9.4 | 3.0 | 86.9 |

### Example 4

Example 4 demonstrates another method for preparing a modified aluminum chloride catalyst. .

A round bottom flask was fitted with a −80° C. condenser, purged with Ar, and 10 g (75 mmol) of AlCl₃ (Aldrich-99% pure) was added and stirred mechanically, also under Ar. Keeping the temperature below 65° C., 35 ml (about 52 grams or 380 mmol) of CFCl₃ was added over a 1.5 hour period. The resulting suspension was stired an additional 3 hours while volatiles (CF₂Cl₂) were allowed to escape through the warmed condenser. The condenser was then replaced with a simple stillhead, and most of the CCl₄ was distilled under reduced pressure. Finally, the last traces of volatiles were removed by warming the residual solid to 30° C.-35° C. at 0.5 mmHg pressure for ˜6 hours, until condensate stopped appearing in the cold trap (maintained at −78° C.). Portions of the catalyst were weighed under Argon blanket as needed.

### Example 5

Example 5 demonstrates a reaction of CH₂F₂ with CF₂═CF₂ to produce CF₃CF₂CH₂F by using a modified aluminum chloride catalyst prepared according to the procedure described above in Example 4. Difluoromethane 16 grams (0.307 mol), aluminium chlorofluoride catalyst 1.5 grams and 45 grams of TFE (about 0.45 mol) were added to a 200 mL stainless steel shaker tube. The reaction mixture was agitated at 65-70° C. for 12 hours. The reaction products were condensed in a trap, then distilled to yield CF₃CF₂CH₂F (15 g, 32%), b.p. 0-1° C. The residual catalyst was black.

### Example 6 (Comparative)

Example 6 demonstrates that no reaction occurs between CH₂F₂ and CF₂═CF₂ when TaF₅ is used as a catalyst under conditions similar to Examples 2 and 3 above.

A 400 ml Hastelloy C shaker tube was charged with TaF₅ (8 g, 0.029 mol) . The tube was cooled down to −30° C. and evacuated. The tube was then charged with CH₂F₂ (26 g, 0.5 mol) and CF₂═CF₂ (40g, 0.4 mol). Then the reaction mixture was warmed up to 100° C. and stirred at 100° C. for 8 hours. No reaction was detected.

### Example 7

Example 7 demonstrates a method for preparing a modified aluminum bromide catalyst.

A 1 L four-neck flask was equipped with an addition funnel, a mechanical stirrer, a dry ice condenser and nitrogen purge. The flask was charged with AlBr₃ 13.33 g, 0.05 mol) and 100 ml of perfluorohexane. CCl₃F (34.3 g, 0.25 mol) was then slowly added to the flask under nitrogen purge through the addition funnel in 70 minutes. Then reaction mix was stirred at 15 to 18° C. for 3 hours after all CCl₃F had been added. The volatiles were removed in vacuum and the resulting modified aluminum bromide catalyst was dried under vacuum at room temperature.

### Example 8

Example 8 demonstrates a reaction of CH₂F₂ with CF₂═CF₂ to produce CF₃CF₂CH₂F by using a modified aluminum bromide catalyst.

A 400 ml Hastelloy C shaker tube was charged with 5 g of modified aluminum bromide prepared according to the procedure described above in Example 7. The tube was cooled down to −20° C. and evacuated. The tube was then charged with 23.4 g of HFC-32 and 30 g of TFE. Then the reaction mixture was stirred at 60° C. for 8 hours. 52.3 g product mixture was collected in a cold trap and was analyzed by GC-MS. The analytical results are given in units of GC area % in Table 3 below. Small amounts of other products, having GC area % less than 0.1, are not included in Table 3.

TABLE 3

| CH₂F₂ | CF₂ = CF₂ | CF₃CHF₂ | CF₃Br | CF₃CF₂CH₂F | CHF₂CF₂CF₂Cl | CF₃CF₂CH₂Br |
|---|---|---|---|---|---|---|
| 43.3 | 46.2 | 0.1 | 0.2 | 7.8 | 0.4 | 0.4 |

As seen in the above examples, the processes herein disclosed produce a hydrofluorocarbons directly without the need for a separate halogen exchange step of the prior art. In addition, even when the catalyst is generated in situ, significant amounts of the desired hydrofluorocarbon is produced, simplifying any subsequent separation procedures.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination. Further, reference to values stated in ranges include each and every value within that range.

What is claimed is:

1. A process comprising: reacting
a hydrofluoromethane which is selected from the group consisting of CH₂F₂ and CH₃F, with
a fluoroolefin which is selected from the group consisting of CF₂═CF₂, ClFC═CF₂ and CF₃CF═CF₂, in the presence of
an aluminum catalyst to produce a hydrohalopropane or a hydrofluorobutane.

2. The process of claim 1 wherein said aluminum catalyst is AlF₃.

3. The process of claim 1 wherein said aluminum catalyst is selected from the group consisting of modified aluminum chloride, modified aluminum bromide and mixtures thereof.

4. The process of claim 3 further comprising: recovering said hydrohalopropane or said hydrofluorobutane.

5. The process of claim 3 wherein said catalyst is produced before said catalyst is contacted with said hydrofluoromethane or said fluoroolefin.

6. The process of claim 3 wherein said catalyst is produced in situ.

7. The process of claim 3 wherein said hydrofluoromethane is CH₂F₂, said fluoroolefin is CF₂═CF₂, and said hydrohalopropane is CF₃CF₂CH₂F.

8. The process of claim 3 wherein said reaction temperature is from about −10° C. to about 200° C.

9. The process of claim 3 wherein said reaction temperature is from about 0° C. to about 100° C.

10. The process of claim 3 wherein the mole ratio of said hydrofluoromethane to said fluoroolefin is at least 1:1.

11. The process of claim 10 wherein the mole ratio of said hydrofluoromethane to said fluoroolefin is at least 3:1.

12. The process of claim 11 wherein the mole ratio of said hydrofluoromethane to said fluoroolefin is at least 5:1.

13. The process of claim 3 wherein a solvent is used in the reaction process.

14. The process of claim 13 wherein said solvent is said hydrofluoromethane.

15. The process of claim 13 wherein said solvent is said hydrohalopropane.

16. The process of claim 13 wherein said solvent is said hydrofluorobutane.

17. A process for producing hydrohalopropane or hydrofluorobutane comprising:
1) providing an aluminum catalyst in a reaction vessel, said catalyst is selected from the group consisting of AlF₃, modified aluminum chloride, modified aluminum bromide, and mixtures thereof,
2) providing in said reaction vessel a hydrofluoromethane which is selected from the group consisting of CH₂F₂ and CH₃F, and a fluoroolefin which is selected from the group consisting of CF₂═CF₂, ClFC═CF₂ and CF₃CF═CF₂, and
3) reacting said hydrofluoromethane and said fluoroolefin in the presence of said catalyst to produce a reaction product selected from the group consisting of hydrohalopropane and hydrofluorobutane,
wherein the reaction temperature is between 0° C. and 100° C., the molar ratio of said hydrofluoromethane to said fluoroolefin is at least 1:1 and wherein the reaction takes place in an inert solvent comprising said reaction product.

18. The process of claim 17 wherein the step of providing a catalyst further comprises: reacting an aluminum halide with CCl₃F, and removing the volatile reaction products to produce said aluminum catalyst.
